(19) United States
(12) Patent Application Publication
(10) Pub. No.: US 2019/0387375 A1
(43) Pub. Date: Dec. 19, 2019

(54) SYSTEM AND METHOD FOR FOOD QUALITY MONITORING AND INTELLIGENT RESTOCKING

(71) Applicant: Candibell, Inc., Andover, MA (US)

(72) Inventor: Lunan Gao, Andover, MA (US)

(73) Assignee: Candibell, Inc., Andover, MA (US)

(21) Appl. No.: 16/137,835

(22) Filed: Sep. 21, 2018

Related U.S. Application Data
(60) Provisional application No. 62/684,770, filed on Jun. 14, 2018.

(51) Int. Cl. H04W 4/35; G01N 33/02; G06K 19/077; G06K 19/07; G06Q 10/08

(52) U.S. Cl. CPC H04W 4/35; G01N 33/02; G06Q 10/087; G06K 19/0717; G06K 19/07758

(57) ABSTRACT

Advanced automated monitoring systems and methods that monitor food quality in real time, and alert consumers for signs of food quality deterioration based on various environmental and biochemical sensor inputs, and knowledge of the food and its holding requirements. The system uses sensor data and rules to estimate the rate at which food is being consumed and may make predictions on when the food will be fully consumed. The system may promptly notify the consumer of when the food will be depleted or spoiled and optionally may issue instruction(s)/message(s) to place an order to replenish and restock the food.

### FIELD

The present disclosure relates to food quality and safety monitoring.

### BACKGROUND

Food spoilage is a common problem facing everyone every day, for all types of food, perishable and non-perishable. Deterioration causes food to lose quality, nutrition and taste. Unconsumed food is wasted and needs to be removed immediately from its holding environment. Increased levels of bacteria and microorganisms developed during the deterioration process can cause various foodborne illness, if not detected promptly and prevented from being eaten.

The difficulties of managing food spoilage are multifold. To start with, there lack ways for open and consistent labeling over food's entire life cycle, from retail to consumer holding, and to and over its consumption. When food is brought home, consumers must read the package themselves to obtain the expiration date and learn about the storing requirements. This manual process greatly hinders the ability for automated food quality monitoring and alerting, leaving great room for error and waste.

Besides difficulties of setting up a monitoring system with food specific storing information, the time it takes for food to expire is a complex function of food type as well as environmental conditions over its holding period. In most cases, the equipment needed to perform such monitoring is found only in a laboratory situation and not accessible to everyday consumers due to the cost, complexity, and consumers' lack of knowledge to operate such equipment.

Food quality monitoring is a continual process over time. It is impossible for consumers to attend to the monitoring only at sparse sampling points without the knowledge of the entire history of the holding environment.

### SUMMARY

The present disclosure provides advanced automated monitoring systems and methods that monitor food quality in real time, and alert consumers for signs of food quality deterioration based on various environmental and biochemical sensor inputs, as well as knowledge of the food and its holding requirements. The system and method are designed to monitor multiple foods at the same time.

Using various sensor inputs, including inputs relating to a consumer's gestures, as well as orientation of the food container, combined with data on the consumer's food consumption history, and dietary pattern, the system uses particular rules to estimate the rate at which food is being consumed and may make predictions on when the food will be fully consumed. The system may promptly notify the consumer of an appropriate consumption date/time and optionally may issue instruction(s)/message(s) to place an order to replenish and restock the food.

Prior to placing a food inside a storage environment such as a refrigerator or a pantry, the system allows a consumer to affix a sensor system to the food, its package or container. Each sensor system, hereinafter referred to as a “label” may be configured with the attributes and holding requirements of the target food. For example, if the target food is a dairy product such as milk, information regarding the target food's holding time at various holding conditions are loaded onto the label or a connected server. Examples of various holding conditions may include optimal range of temperature, humidity, and light intensity.

Additionally, the label may be configured with sensors specific to the food type being monitored. The label may be affixed to the food package, e.g. a milk bottle, before it is placed into the storage system. Alternatively, a pre-defined label specific to the food type to be monitored may be used. Each such pre-defined label is pre-loaded with information about specific food requirements eliminating the need for the consumer to manually input the information. For example, a milk label may be pre-loaded with sensors specific for monitoring milk as well as information regarding milk's holding time at various holding conditions such as optimal range of temperature, humidity, and light intensity.

Depending on the technical configuration implemented between the food source, e.g. a merchant that sells food, and the consumer utilizing the food storage system according to the specialized system and method, there are at least two ways the disclosed system may work.

In a first embodiment, the food source, such as a food merchant, affixes a label to the food or its package. The label contains a condensed label, such as a barcode, QR code, RFID or the like comprising the identity of the food as well as relevant information on its storage requirements and an ideal set of holding parameters. The label can be conveniently read by a consumers' storage system such as a refrigerator, or, drawer, closet or food storage box with the help of a reader, and the information from the label may be processed by a specially configured processor. When foods with a label are placed into the consumer's food storage system, the label may be read allowing the storage system to obtain the food's expiration and optimal storage requirements. Before food expires, or when the sensors inside the storage system detect deviation from the ideal environment as specified by the label, an alert may be sent to the consumer.

In an alternative embodiment, the system may allow the consumer to affix the consumer's own labels for monitoring food quality. Each label may be configured with the properties and holding requirements of the target food. This configuration step may be omitted if pre-defined labels for a specific food are used. Each such pre-defined label already contains information about the specific food it targets and includes the relevant sensors for monitoring of the food. The pre-defined label may be re-usable. Consumers may then directly associate a matching pre-defined label to a food that needs to be monitored in the food storage system. Each label may optionally contain environmental and biochemical sensors that help detect deviation from food's ideal holding environment, as well as signs of deterioration.

This has outlined, rather broadly, the features and technical advantages of the present disclosure in order that the detailed description that follows may be better understood. Additional features and advantages of the present disclosure will be described below. It should be appreciated by those skilled in the art that this present disclosure may be readily utilized as a basis for modifying or designing other structures for carrying out the same purposes of the present disclosure. It should also be realized by those skilled in the art that such equivalent constructions do not depart from the teachings of the present disclosure as set forth in the appended claims. The novel features, which are believed to be characteristic of the present disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

Embodiments of devices, systems, and methods are illustrated in the figures of the accompanying drawings which are meant to be exemplary and non-limiting, in which like references are intended to refer to like or corresponding parts, and in which:

FIG. 1 illustrates the components of the disclosed system when configured to use a shared label between a food merchant and a consumer's food storage system;

FIG. 2 illustrates the components of the disclosed system using a consumer-affixed label and sensors for food quality monitoring;

FIG. 3 further illustrates components of the disclosed system;

FIG. 4 illustrates a process flow diagram of a system to monitor food consumption and restock the food;

FIG. 5 illustrates technical interaction among the components of the system configured to monitor food conditions and restock the food;

FIG. 6 is a block diagram illustrating the components of a label in the system configured to monitor food conditions;

FIG. 7 illustrates an embodiment of a label configured to monitor food and environmental conditions;

FIG. 7a illustrates an embodiment of the label for monitoring food quality with a fastener for affixing the label to a food;

FIG. 7b illustrates another embodiment of the label for monitoring food quality that uses a retractable leash for affixing the label to a food;

FIG. 7c illustrates still another embodiment of the label for monitoring food quality that is in a clip form factor for affixing the label to a food;

FIG. 8 is a block diagram illustrating components of a hub configured to receive communications and sensor data from a label for monitoring food conditions;

FIG. 9 illustrates an example embodiment of a process flow diagram detailing a method for a system to monitor food conditions using the components of the hub;

FIG. 10 illustrates a process of provisioning a label and a hub to a consumer account;

FIG. 11 illustrates a method of forming and updating a quality model and a rule set for an expiration alert for the method and system to monitor food conditions.

### DETAILED DESCRIPTION

The detailed description of aspects of the present disclosure set forth herein makes reference to the accompanying drawings, which show various embodiments by way of illustration. While these various embodiments are described in sufficient detail to enable one skilled in the art to practice the disclosure, it should be understood that other embodiments may be realized and that logical and mechanical changes may be made without departing from the spirit and scope of the disclosure. Thus, the detailed description herein is presented for purposes of illustration only and not of limitation. For example, the steps recited in any of the method or process descriptions may be executed in any order and are not limited to the order presented. Moreover, references to a singular embodiment may include plural embodiments, and references to more than one component may include a singular embodiment.

The present disclosure relates generally to a specially configured food quality and safety monitoring and intelligent restocking system and method in the broad and emerging area of smart home and home automation. The system may be configured to receive food quality information from a label and store the information on a server. The server is then able to track food consumption and determine whether a food will spoil and expire, or be fully consumed. In either case, the system may alert a consumer to replenish or restock the food. Alternatively the server may send a request to a merchant determined by the consumer to restock the food. It will be appreciated that the terms “spoil” or “expire” are used generally to indicate that the food is near or past its edible condition and may be used interchangeably.

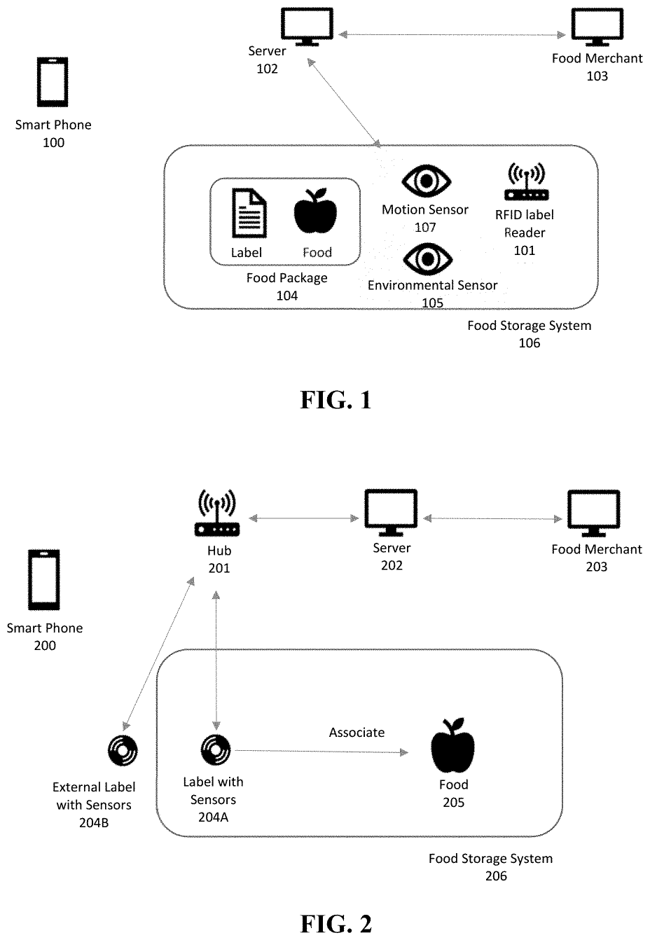

FIG. 1 illustrates the components of the disclosed system when it is configured to use a shared label between a food merchant 103 and a consumer's food storage system 106. Food in a package 104 is prepared and sold by a food merchant 103. The food package 104 contains a label affixed to the food or its package. The label contains information about the food itself, including the name, category, preparation guidelines and expiration time, as well as the desired parameters for an ideal holding environment. The label can be wirelessly read by a remote label reader 101 placed inside the food storage system 106. The label reader 101 is configured to understand the communication protocol with the label from the food package 104 as well as its format and content. When the food package 104 is placed into the food storage system 106, a reader 101 inside the food storage system 106 may detect the presence of the food package 104 and retrieve information from the label about the food and its holding requirements wirelessly, without additional user input. Alternatively, a hardline connection may be established between the label and the reader 101 for retrieving information from the label.

Once the reader 101 obtains the information, it may share the information with the food storage system 106. The food storage system 106 may be equipped with an array of sensors, including motion sensors 107 and environmental sensors 105 such as biochemical sensors, to monitor the food storage system's 106 environmental factors such as temperature, pH level, humidity, lighting condition, as well as biochemical characteristics relevant to the target food and category. When sensors 105 detect deviation in the environmental conditions of the food storage system 106 from the food's ideal holding requirements, or signs of food deterioration in the food, the sensors 105 may notify the specially configured processor associated with the food storage system 106, which may send an alert to the server 102. The server 102 may forward the alert to a consumer's smartphone or device 100 to display the alert. A food storage system 106 can also send reminders to the consumer, via the same path, regarding if the food is about to expire or the best time to consume the food to achieve a balanced nutrition intake, taking into consideration the consumer's diet history and other information about the consumer and the consumer's dietary goals. The food storage system 106 may also be configured with a set of motion sensors 107 that may detect when the food is consumed and how much of the food remains correlated with a date/time monitored by the specialized processor associated with the food storage system 106. This information is useful for predicting when the food is running out and needs to be replenished. The rule set that is implemented to perform such prediction may maintain a model that is self-calibrating. The rule set can either run on the food storage system 106 processor or on a server 102.

If the food needs to be replenished, the server 102 may push a message to the consumer's smartphone or device 100 to let the consumer know that the food needs to be replenished. The consumer may handle the purchase or, the system, based on the consumer's earlier settings, may directly order the food from the food merchant 103. The consumer may pick up the food or the food may be delivered to the consumer's address stored on a database located on the server 102 or with the food merchant 103. The food merchant 103 that sells the food initially, may or may not be the merchant that receives the replenishment order. The decision of which merchant receives the order may depend on the consumer's brand and/or loyalty preference, location and distance, real-time pricing information, or food availability. The food storage system 106 may work with the server 102 to keep track and monitor multiple instances of food in different packages 104 at any time. FIG. 1 is an example that depicts the situation when only one food is being monitored. It should be appreciated by one skilled in the art that such a system may be configured to monitor multiple foods without deviating from the above disclosure.

FIG. 2 illustrates components of an alternative embodiment in which a consumer uses separately purchased labels with sensors for food quality monitoring and restocking. The key difference in the embodiment of FIG. 2 compared with FIG. 1 is that food sold by a food merchant 203 in FIG. 2 may not include a label with information regarding the food's expiration and holding requirements that are machine-readable by the food storage system 206. To automate the monitoring process, the consumer may manually affix a label 204A to the food 205 before placing the food 205 within the food storage system 206. A consumer may first configure the label 204A with information specific to the target food 205. This may include, but is not limited to, food name, category (e.g. fruit, meat, dairy), estimated expiration date, requirements for an ideal holding environment, and/or the best time to consume the food 205.

The configuration of the label 204A may require the consumer to use a smartphone or device 200 to communicate with and setup the label 204A such as with an RFID programming application. Next, the consumer associates the label 204A with the food target 205. The association can take different forms depending on the specific shape and design of the label 204A, the food, or the food's packaging. For example, a label 204A can be clamped, strapped or glued, or otherwise attached onto the package of the food 205, or it can be placed on top of the food 205.

The label 204A, in addition to being equipped with an array of environmental and biochemical sensors, may also be equipped with motion sensors (e.g. accelerometer or gyroscopic micromechanical devices) that are capable of detecting a consumer's motions relating to consuming a food 205, such as lifting up the food container, tilting bottles, or other complex movements or gestures with the food or its package 205 with such sensors attached or located proximate to the food 205 to monitor the movement of the food or its package.

It should be appreciated that not all labels need to be placed inside a food storage system 206. An alternative label 204B may be configured to monitor the food 205 from a location external to the food storage system 206 and without any sensor requiring physical contact with the food 205. For example, if a food storage system 206 is a refrigerator, the label 204B can take the form of a sticker or magnet for placing on a refrigerator door or exterior panel. A consumer may place a food 205 into the food storage system 206 and begin a monitoring session using label 204B. The label 204B may include a timer which may be started when the food 205 is stored in the food storage system 206. When the food is consumed, the consumer may stop the timer on the label 204B. The start and stop of the timer, as well as the start and stop of the monitoring session of the food, does not necessary require a definitive gesture or action from the consumer such as a button press. The label 204B may detect a change in its location, proximity, movement, or a variation in environmental temperature to intelligently determine when to start and stop the monitoring session. The label 204B may also be configured to associate with sensors internal to the food storage system 206 for additional monitoring of the food 205. Additionally, a motion sensor 107 integrated into the labels 204A and 204B may detect the proximity of a consumer for determining whether an indicator on the labels 204A and 204B should turn on to notify the consumer of the condition of the monitored food 205.

If the food is about to expire, an alert may be generated and wirelessly transmitted to a hub 201. The wireless protocol can be a standard 2.4G Bluetooth protocol, or other wireless standards such as Zigbee and Z-Wave. The labels 204A and 204B may be identified when they communicate with the hub 201 using a unique identifier. The hub 201 may store a provisioning list identifying which labels belong to the consumer, and may only monitor communications with the consumer's labels and ignore other labels present. A provisioning model discussed hereinafter, allows the consumer to specify a list of labels that are registered to the consumer's account by using a smart phone or device 200. The list of provisioned labels may be synchronized between a server 202 and the hub 201. The smartphone or device 200 may have network connectivity with the server 202. The hub 201 may also have network connectivity with the server 202, in addition to its connectivity with the labels. The connectivity between the labels and the hub 201 may be a wireless connection or a physical connection.

The labels may be equipped with sensors including motion sensors similar to the motion sensor 107 of FIG. 1. These sensors may help predict when the target food 205 becomes depleted as a result of user consumption, using information regarding the food 205, a consumer's historical consumption data, and sensor inputs that estimate the rate at which the food 205 is being consumed. The time a replenishment is needed is therefore the earlier event of 1) when the food becomes expired, or 2) when the food becomes depleted. If a replenishment or restocking of the food 205 is needed, the server 202 may notify the consumer on their smart phone or device 200. The server 202 may also place an order with a food merchant 203. The consumer may pick up the order from the food merchant 203 or the food merchant 203 may be notified to have the order delivered to the consumer's address.

In this embodiment, the consumer may need to configure the label 204A or 204B prior to a monitoring session. To save effort and optimize user experience, a set of pre-defined labels can be offered to the consumer. Each pre-defined label 204A or 204B may work for a specific food 205 or food category. For example, pre-defined labels for milk, for fresh beef, or for strawberries can be prepared in advance for use in monitoring a food 205. In this case, the consumer may directly attach a pre-defined label 204A to a corresponding food 205 while loading the food 205 into a food storage system 206. Alternatively, a pre-defined label 204B can be located external to the food storage system 206 during a monitoring session. The pre-defined labels aid the consumer by eliminating the need to first connect to and configure a general purpose label.

The food monitoring labels 204A and 204B may be further configured to send sensor data to the hub 201 and receive instructions from the hub 201. The hub 201 may be located near or within the food storage system 206 and may be configured to receive the sensor data from the food monitoring labels. Upon receiving the sensor data, the hub 201 may forward the sensor data to the server 202. The hub 201 may also be configured to receive instructions from the server 202 and forward the instructions to the food monitoring labels. The food monitoring system may additionally have a storage panel (not shown) for holding the food monitoring labels when not in use. The storage panel may be designed with a fastener configured to receive the food monitoring labels, a charger configured to supply power to the food monitoring labels for charging a battery, and a LED for notifying the consumer of the battery level and charging status of the labels.

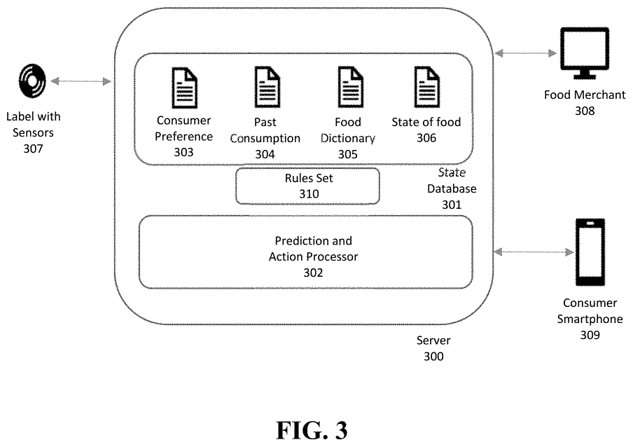

FIG. 3 further illustrates components of the disclosed system. A server 300 consists of one or more database(s) 301, a rules engine and rules set 310 and a prediction and action processor 302. A state database 301 keeps track of a set of food data 306 with the food holding environment history since the food 205 was stored. The state database 301 may also include a food dictionary 305 which contains a list of foods and food types with the relative time to expiration, as a function of the food's holding environment, as well as specific measures and thresholds used for detecting signs of deterioration in the specific food and food type being monitored. For example, the food dictionary 305 may be a look-up table that contains information regarding measures and thresholds for detecting deterioration in fruit as well as the time for a fruit such as a strawberry to expire in different holding environments.

A past consumption storage database 304 may be a database or database segment that records the quantity of food consumed in each period or shopping cycle of the consumer. A consumer preference storage database 303 may store a consumer's dietary goals such as caloric and nutrition intake targets, popular food and food types of a consumer and the habitual stored quantity at the beginning of each storage cycle, preferred merchants, conditions on automatic replenishment, as well as other preferences that can affect prediction or action by the prediction and action processor 302.

The sensors 307 provide various sensor readings on the holding environment and relevant biochemical characteristics of a target food 205. This data may be provided to the state database 301 to update the current state of the food 306. In cases where information regarding consumption is provided, such as when a consumer lifts up a food package or the consumer resets the labels, indicating food is fully consumed, the past consumption database 304 is updated. In an embodiment, a food merchant 308 may also upload information/data to the state database 301 by providing the food merchant's 308 food supply information such as availability, location, current promotion, pricing, and time to deliver.

The information from the state database 301 may be processed according to the rules engine reading the rule set(s) 310 via a prediction and action processor 302 which then yields various predictions and recommended actions. Predictions and recommended actions from the processor 302 may consist of time remaining to food expiration, suggesting a meal meeting the consumer's nutritional needs using available food in the food storage system 206, suggesting to replenish the monitored food 205, suggesting a shopping list based on the availability and quality of food in the food storage system 206 as well as the availability and pricing of the food 205 from the food merchant 308. The consumer smartphone or device 309 may receive notifications from the server 300 regarding predictions and actions generated by the processor 302. The consumer device 309 may also allow the consumer to view and update information from/to the state database 301.

If the sensor data from the labels/sensors 307 is noisy (e.g., value sampled and observed deviate from their true values), the system may suffer from missing data or non-precise observations from time to time. The rules set 310 may take this imperfection into consideration and propagate this imperfection, or variance, over itself and provide a set of “best effort” predictions and/or suggested actions. The rules set 310 may be self-calibrating, correcting itself continuously as the system gathers data from sensors and issues predictions and actions using artificial intelligence mechanisms known in the art.

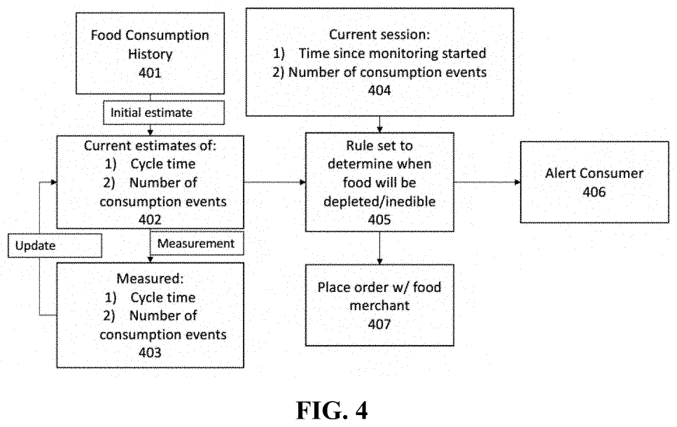

FIG. 4 illustrates a process flow diagram detailing a model for a system to monitor food consumption and restock. The model may keep track of and accurately estimate the rate at which a food 205 is consumed in order to improve the model's prediction 402. Initially, the model may be fed with prior data and behavior preferences collected 401 from the consumer. This information 401 may include, but is not limited to: household size, quantity/amount for each food type purchased per shopping cycle, and how long it takes the consumer to fully consume the stored quantity/amount. As the model runs, it may keep track of and estimate the cycle time 402 for each type of food, as well as how many consumption events are needed to fully consume the quantity/amount obtained in a shopping cycle 403.

The cycle time may be measured or calculated 403 using the time period from the start to the end of a monitoring session using the labels 204A and 204B. The number of consumption events can be captured and estimated 402 using motion sensors 107. A rule set 405 may be used to determine when food 205 will likely run out and need to be replenished, based on how long it has been since the target food 205 was last added to the storage system 206, as well as how many consumption events have occurred in the current monitoring cycle 404. The model may also be programmed to predict when a food 205 will run out and order replenishment and restocking ahead of time. If a type of food 205 is deemed to run out soon, the model may send an alert to the consumer 406 and display a prompt for the consumer to place an order 407 for the food 205 from a food merchant 203 using a smartphone or device 200.

As an example: a User is single and uses a label to track consumption of eggs. Without any prior information, the tracking system assumes a cycle time of 10 days, with a total number of consumption events of 10 for single user. This averages to consumption rate of 1 consumption event per day. As the User starts using a label to track egg consumption, the system estimates the User's actual habitual consumption rate by measuring:

Time it takes between tracking starts and tracking ends for the first few tracking sessions;

Consumption events as indicated by onboard motion sensor in the label for each session;

In this example, the system found that average consumption rate is higher: with average cycle time of 5 days, and average number of total consumption events per cycle of 10. This translates to 2 consumption events per day. During an active tracking session, the system found itself 2 days into the tracking session, with recorded consumption events of 6. The predicted time remaining for the User to run out of eggs is estimated as:

(Average total number of consumption events−6)/consumption rate

(10−6)/2=2 days,

therefor the system estimates, given the prior habitual information and how the current session runs, another 2 days until the User runs out of eggs.

Although the sensor data may be noisy and non-perfect, the model may be configured to constantly self-calibrate 405. In one embodiment, when a consumer ends each monitoring session, the model counts the time elapsed from the beginning to the end of the monitoring session and the number of consumption events 403. These two measurements may be used to correct the prior estimates 402 of the two parameters; for example, by taking the average of the estimated and measured values and using the average as the new estimate 402 for the next monitoring and prediction cycle. Using the example above, every time when the User tracking is finished, the system obtains a new recorded cycle time and total number of consumption events. The two observed values are respectively averaged with their prior values to produce an updated pair of estimates for prediction in a future tracking session.

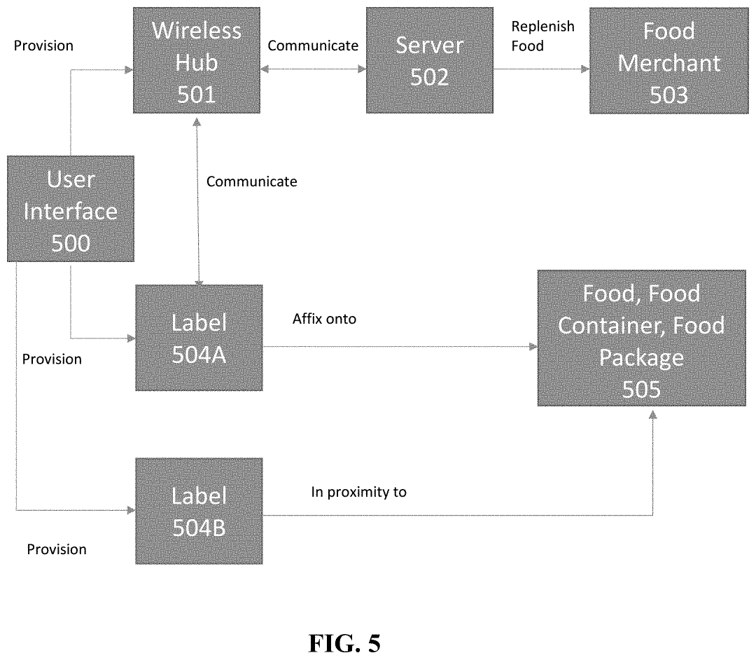

FIG. 5 illustrates technical interactions among the components of the system configured to monitor food conditions and restock the food. In the illustration a consumer manually affixes a label 504A to the food 505 before placing the food 505 into a food storage system 206 such as a refrigerator, a drawer or a closet. The label 504A may be configured with the name and category of the affixed food 505. The configuration process may allow a consumer to use a user interface such as an application on a smartphone or device 500 to communicate with and configure the label 504A using wireless technology. Alternatively, a hardline connection may be established to configure the label 504A. The connection between the label 504A and the food 505 can take different forms depending on the specific shape, material, and exterior design of the label 504A, the food and the food's package/container 505.

For example, a label 504A can be clamped or affixed onto the package of the food 505, or it can be placed simply on top of the food 505. Alternatively, a label 504B may be configured to monitor a food 505 without physical contact using, for example, optical and odor sensors. Sensors/labels may be made available as a package containing multiple sensors/labels 504A and 504B. Such a package may include a set of labels that monitor the holding environment and detect signs of deterioration in the food 505. These sensors are of a wide variety and may include a temperature sensor, humidity sensor, air quality sensor such as a CO2 and Total Volatile Organic Compounds (TVOC) sensor, photoelectric and light sensor, color sensor, water sensor, optical sensor, and biochemical sensors or combinations thereof and the like. Each of the labels 504A and 504B may be equipped with an independent temperature sensor as it may often be necessary to have a dedicated temperature sensor for each food target 205, even if multiple targets share a common storage space 206. Each sensor monitors a particular set of attributes of the target food 505 while inside the storage environment 206. The labels 504A and 504B may include all of the sensor types, or any subset of them, depending on the labels' purpose and food target 505. A set of pre-defined labels, designed to be easily identifiable, for monitoring a specific type of food 505 may be provided to the consumer. Each of the pre-defined labels may be equipped with sensors to target the relevant characteristics for a particular food type such as dairy, beef/chicken/meat, vegetable and the like. Although food has been described as a food, one skilled in the art should appreciate that the labels may also be used to monitor beverages such as milk, juice, and other goods such as condiments, salad dressings, and the like.

In addition to monitoring the holding environment 206 and quality of the food target 505, the labels 504A and 504B may also be equipped with sensors or an array of sensors for detecting movement of the food 505, such as being lifted up, placed down, tilting, rotating the food container or package 505, as well as other complex movements of the food or its container 505. These motions and movements are strongly correlated with events of food consumption and may help the system estimate the current rate of food consumption, and predict, based on historical information and habitual behavior of the consumer, the projected time at which the food 505 will likely run out. This enables the food monitoring system to better manage the food inventory by projecting when a food 505 will need to be replenished. The system may additionally generate an automatic shopping list and place an order for replenishing and restocking the food 505 automatically with a merchant 503.

The motion sensors installed in the labels 504A and 504B may include but are not limited to: an accelerometer, hall sensor, proximity sensor, touch sensor, pressure sensor, gyrometer/gyroscope and compass, laser range finder, infrared IR distance sensor and/or flow sensor, and ultrasonic sensor. The labels may be pre-defined for a specific food type and may include only a subset of the motion sensors for detecting consumption of the food type.

In an illustrative embodiment, a consumer may associate at least one of the labels with a food 505, the label(s) is then instructed to start a monitoring session. When the food 505 is fully consumed or degraded to the point it must be removed from the storage system 206, the label(s) may be instructed to end the monitoring session. The start and stop of the monitoring session of the label(s) may require a definitive gesture or direct command from the consumer by way of interacting with a user interface 607, such as a voice command/control, an actuator or a button on the label(s), sending a command to a server 502 monitoring the label, and/or interacting with the consumer's smartphone or device 200 using an application, or interacting with a hub 501 connected with the label(s). The label(s) may detect, based on the sensor observations, changes in the label's location, proximity to the food target 505, movement of the food, and variation of surrounding temperature, magnetic field and its orientation and intensity to intelligently determine the beginning and end of a monitoring session.

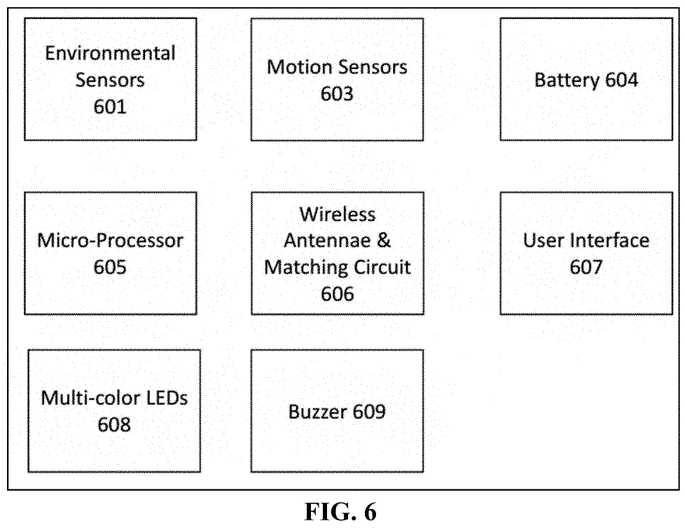

FIG. 6 is a block diagram illustrating components of a label 204A and 204B (or 504A and 504B). The label is a computing device which monitors a food 205 and/or the environmental conditions of a food storage system 206. The label may be a small, battery 604 powered computing device which communicates wirelessly with a hub 201 and reports on its sensor observations. The label may include a user interface 607 including one or more actuators, such as a button, allowing a consumer to issue instructions to the labels. Various patterns of button press may be used to issue instructions to the label by the consumer, such as start, stop, or pause a monitoring session, turn on and off the label's main power, or enable the label to perform a Bluetooth pairing.

A micro-processor 605 is included to process and format sensor data and report on the observation with the hub 201. The micro-processor 605 may also encode and decode a wireless communication with the hub 201 using a wireless antennae and matching circuit 606. At least one multi-color LED 608 may be used to provide a visual indication on the progress of food deterioration based on a color coding rule 310. The multi-color LED 608 can be managed, either by logic programmed into the label, or remotely by the server 202, to indicate food quality based on sensor information received from the label. For example, a green color indicates healthy and fresh, while red means do not consume. A yellow LED color may indicate a food is nearing spoilage as predicted by a tracking model (e.g. within 5 days in such a holding environment). To save battery, the LED 608 may only be turned on for a period of time when the consumer is holding the food 205 or in close proximity to the food 205 (with proximity being measured, for example, by one of more sensors onboard the label).

In an illustrative embodiment, the label is positioned inside a refrigerator, upon the consumer opening the refrigerator there is a sudden increase in light intensity impacting a light sensor and alerting the label to the presence of the consumer. In response, the label may illuminate a LED 608 with a color based on the color coding rule 310. Likewise, a detection of movement by the label is a strong indication of the consumer moving the food or its container 205, and therefore of the consumer being in close proximity to the food 205. Upon determining that the consumer is in close proximity to the food 205, the LED 608 may illuminate to alert the consumer to the status of the food 205.

A buzzer or sound actuator 609 may be programmed to ring based on a rules set 310, providing audio feedback reflecting the food quality level. For example, a mid-pitch tone may indicate that food is in a consumable state and a high pitch tone may be used to alert the consumer to possible food deterioration. The combination of a LED 608 and a buzzer 609 may be used to provide quick and effective feedback regarding the quality level of the food target 205 when a consumer is in close proximity to the food 205 without requiring the consumer to check with the server 202 for an update on the food's 205 quality.

The labels may include a number of sensors. Environmental sensors 601 may measure various characteristics and attributes regarding the holding environment and the target food 205. The sensor data may be reported to the server 202. The label may report to the server 202 in real time on a continuous basis or at timed intervals. Based on a rules set 310, the server 202 can then estimate the current state of the food 306 and issue alerts and recommendations to the consumer as necessary. Table 1, below, illustrates examples of different environmental sensor types 601 and attributes they may measure. A pre-defined label may include a subset of these environmental sensors.

TABLE 1
Environmental Sensor and Attributes

| # | Sensor Type | Environmental Attribute |
|---|---|---|
| 1 | Temperature | Temperature |
| 2 | Humidity | Humidity |
| 3 | CO2 and TVOC | Air quality |
| 4 | Photoelectric | Level of light exposure and intensity |
| 5 | Color | Change in surface color of food |
| 6 | Water | Water |
| 7 | Optical | Ambient light, absence or presence of |
| | | objective (food target) |
| 8 | Biochemical | pH value gas detection |

The labels may also include motion sensors 603 for detecting movement of food or its container 205 as well as movements in the vicinity of the food or its container 205. Such movements are typically correlated with events of food consumption and can be used to estimate consumption rate and predict when food replenishment is needed. Table 2, below, is a list of motion sensors 603 that may be included in the labels. A pre-defined label may include a subset of these motion sensors.

TABLE 2
Motion sensors and attributes they measure

| | | Measuring | | | |
|---|---|---|---|---|---|
| # | Sensor | attributes | # | Sensor | Attribute |
| 1 | Accelerometer | Multidimensional | 8 | Laser | Presence or |
| | | Acceleration, | | | absence |
| | | Tilting detection | | | |
| 2 | Hall effect | Magnetic field | 9 | Infrared | Distance |
| | sensor | variation | | | |
| 3 | Proximity | Position and | 10 | Ultrasonic | Flow meter |
| | sensor | location | | | |
| 4 | Touch | Touch event | | | |
| | sensor | | | | |
| 5 | Pressure | Touch event, | | | |
| | sensor | weight | | | |
| | | measurement of | | | |
| | | food or its | | | |
| | | container | | | |
| 6 | Gyrometer/ | Orientation, | | | |
| | Gyroscope | tilting | | | |
| 7 | Compass | Orientation | | | |

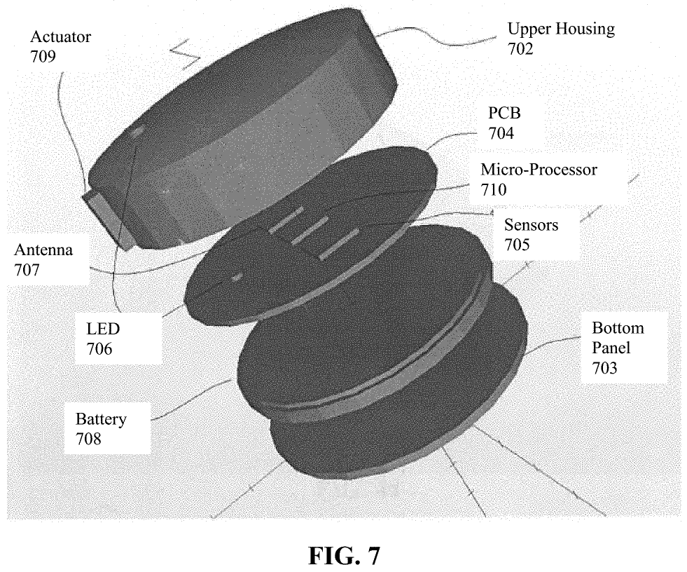

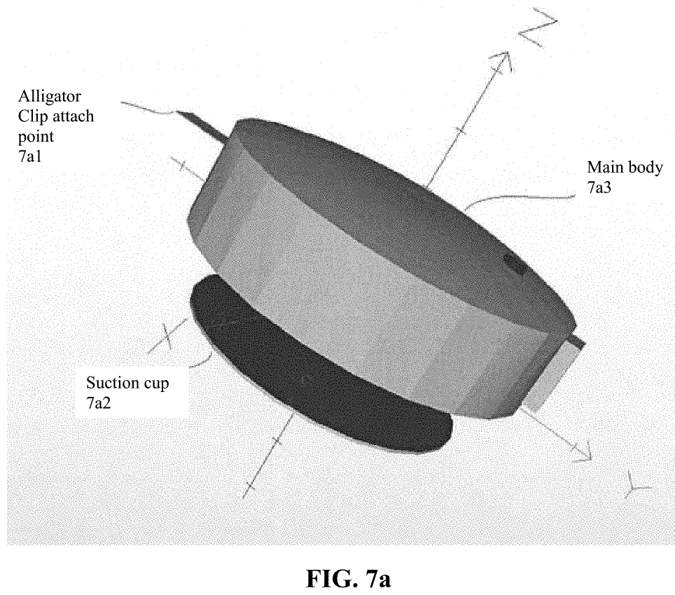

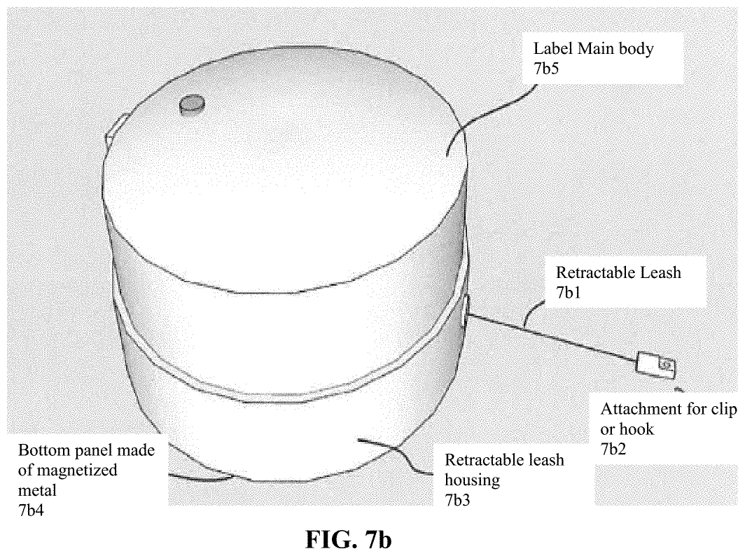

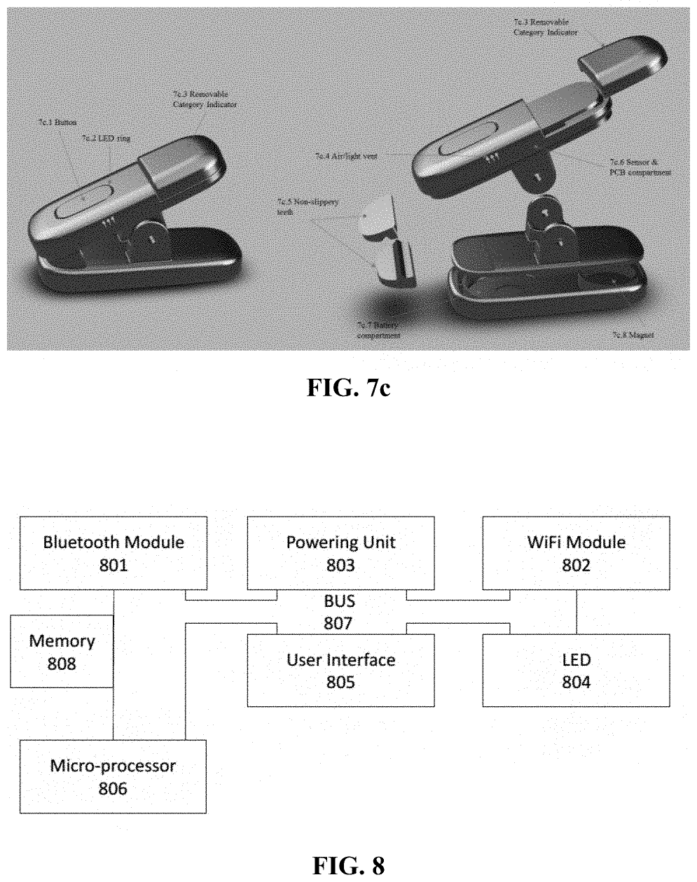

FIG. 7 illustrates an example physical embodiment of a label configured to monitor a food 205 and the environmental conditions of the food storage system. A label may be designed with an upper housing 702 and a bottom panel 703. The upper housing 702 and bottom panel 703 of each label may be finished with food safe material such as BPA-free plastic or a food safe metal coating and sealant. The upper housing 702 may include external decoration and text indicating its pre-configuration, if it is a pre-defined label, for a particular food type. Alternatively, the upper housing 702 may be decorated for aesthetic purposes, e.g. with pictures or symbols of the food type to which it corresponds. The label may house electronic structures including a Printed Circuit Board (PCB) 704, a micro-processor 710, a sensor array 705, at least one LED 706, an antenna 707, a battery 708 and wirings and connectors within the upper housing 702 and the bottom panel 703. The upper housing 702 may host an actuator, such as a button 709, on a side of the upper housing 702, as well as a number of apertures or transparent surfaces for viewing the LEDs 706 as well as for a photoelectric sensor 705 to sample light.

The PCB 704 provides a surface on which to add and connect the sensor array 705 to a power source such as a rechargeable battery 708 as well as a mounting point for the micro-processor 710 and other electronics. The sensors 705 may include environmental sensors 601, motion sensors 603, or a combination of the two. The upper housing 702 and bottom panel 703 may be constructed to provide the sensors access to the target food 205 whether by physical contact or through an aperture to allow a beam such as an optical sensor to monitor the food 205. Additionally, the antenna 707 operatively connects a label to the hub 201 using a wireless connection such as a Bluetooth connection or a WiFi network. The micro-processor 710 instructs the sensors to sample the food target 205 and surrounding environment at set intervals or on a continuous basis and processes the results to be transmitted to the hub 201 through the antenna 707 or a hardline connection between the hub 201 and the label. The bottom panel 703 may be designed to affix the label 504A to the food target 205 or the food storage system 206 using a fastener.

FIG. 7a illustrates an embodiment of a label for monitoring food quality with a fastener for affixing the label to a food target 205. The label may be equipped with a suction cup 7a2 mounted on a bottom panel 703 of the label, with an alligator clip 7a1 mounted on the side of its body 7a3. Each label may include one or more fasteners for attaching the label to a food or its container 205. Such mechanisms may include, but are not limited to, an alligator clip 7a1, suction cup 7a2, hook and pile, a magnet, a retractable leash, hooks, adhesives, or a combination thereof.

FIG. 7b illustrates an alternative embodiment of a label for monitoring food quality using a retractable leash 7b1 for affixing the label to a target food 205. The leash 7b1 may utilize a clip or hook 7b2 to affix to the food 205. The main body 7b5 of a label may be located on top of the retractable leash housing 7b3. The bottom panel 7b4 of the label, may be made of ion-containing metal and thereby attracted to a magnet.

FIG. 7c illustrates a still further embodiment of a label for monitoring food quality. In this embodiment the form factor is a clip. When in use, this label is clipped onto the target food to monitor its holding environment and any motions for consumption events. The clip is composed of an upper housing 7c.6 that houses various environmental and motion sensors such as temperature, humidity, lighting sensors as well as motion sensors such as gyroscope, accelerometer and/or compass. The upper housing 7c.6 also houses a Printed Circuit Board (PCB) or PCBs as well as a micro-processor, a Bluetooth antenna, a matching circuit, a buzzer, power management unit as well as other needed electronic components. The bottom side of the clip includes a battery compartment 7c.7, for an exchangeable battery such as CR2032, and a magnet 7c.8 for attaching to a metallic surface such as a refrigerator. There is a pair of non-slippery teeth 7c.5 attached to the front tip of the clip. On the top of the clip is a button 7c.1 which a user may press, e.g., to indicate the start and end of a tracking session. A multi-color LED ring 7c.2 surrounds the button and may be used to indicate various states the food target is currently in, such as healthy, need to eat now, and discard.

A removable category indicator 7c.3 can slide onto and off from the main body of the clip. The removable category indicator 7c.3 contains an electronic component (e.g. an EEPROM or a resistor network) in which a particular food target is defined using a value. In an EEPROM illustrative embodiment, a food target is defined by a value stored in the EEPROM. If a resistor network is used in place of EEPROM, a food target is defined by the effective resistor network value. The removable category indicator 7c.3 also may have a picture of the food target as well as its name printed on its outer surface that users can read and recognize easily. When a user picks one such category indicator and slides it onto the main body of the clip, the clip recognizes the food target that is stored in the removable category indicator 7c.3 and becomes a clip that tracks for this type of food. If a user wants to track a different type of food using the same clip, the user just needs to remove the current category indicator and slides on a new/different category indicator which is specific to the food target to be tracked. The recognition of food target represented in a category indicator is done through reading the category value, e.g., stored in the EEPROM or the resistor network value. Contact pins are exposed at the bottom of 7c.3. When the removable category indicator 7c.3 is securely installed on a clip, the micro-processor inside the clip can communicate with the removable category indicator 7c.3 via contact pins. The advantages of using a removable category indicator include, among other things, that it has a visible picture and name of the food target that the user can easily see and recognize without opening an app on a phone. Further, this design allows users to re-configure a same clip to track different types of foods by installing a different removable category indicator 7c.3, without the need to interact with a smart phone app, a computer connected to internet or a smart speaker.

The labels described hereinbefore (e.g. 204A, 204B, 504A, 504B, FIG. 7c) may be stored onto a vertical surface such as a refrigerator door or kitchen wall using a fastener to provide easy access and improved consumer experience. An example fastener is a magnet, due to the prevalence of magnetized metal in refrigerator doors. If the surface is magnetized such as those commonly seen on refrigerator door panels, labels with a magnet on the bottom panel 703 may attach firmly to the refrigerator door panel and be retrieved easily.

In some situations, such surfaces do not readily attract magnets, such as tiled kitchen walls, or a stainless-steel refrigerator door. A storage panel may be used when the label cannot fasten to the door or wall on its own. The storage panel may also be used for organizing multiple labels. One face of the panel may fasten to the wall or door using a fastener such as a multi-purpose gel pad or suction cup, while an opposing side may be made of a material that can attract the fastener of a label. Furthermore, the side attracting the fastener of the label may be grid marked, with a magnet embedded beneath the center of each grid. A magnetic field sensor 705 such as a reed switch can be mounted within the bottom panel 703 of a label, the reed sensor may be used to determine if the label is stowed on the storage panel and being attracted by a magnet.

The label's battery 708 may be rechargeable when fastened to the storage panel using a physical connector or wireless charging. As a label is taken off the storage panel, the magnetic field sensor 705 detects the change in magnetic field strength. The label may then start a monitoring session without the need for the consumer to press an actuator, such as a button 709. After monitoring is complete, i.e. the food 205 is consumed, or the food package 205 is to be disposed of or recycled, the label may be returned to the storage panel. The magnetic field sensor 705 may detect the magnetic field change and may send a signal to the hub 201 that the current monitoring session is now complete.

The sensors 705 of the food monitoring labels generate sensor data which may be sent to the hub 201 via an over-the-air connection, or a hardline connection. The hub 201 collects the sensor data then forwards the sensor data to the server 202. The server 202 or the hub 201 may verify the sensor data against a quality model and rules set 310 to determine if the food 205 is in danger of expiring. If the food 205 may expire within a predefined time period (e.g. 5 days) or has expired, an expiration alert 405 for the food 205 may be generated. When an expiration alert 405 is generated by the quality model and rules set 310 the server 202 may notify 406 a consumer regarding the expiration alert 405 if the food quality degrades to being inedible or spoiled.

Alternatively, an expiration alert 405 may be generated if the food 205 is below a threshold amount or has been fully depleted. A threshold amount may be preprogrammed either by a consumer or set as a system default to indicate when the system should place an order for restock and replenishment. Furthermore, the server 202 may be configured to place an order for replenishing and restocking the food with the consumer or with a food merchant 407 when an expiration alert 405 is generated. When a system determines a food 205 is running low and a restocking is needed, the server 202 may notify the consumer on their smartphone or device 200. The server 202 may place an order with a food merchant 203 directly. The consumer may then pick up the order from the food merchant 203 or the food merchants may be notified to deliver the order to the consumer's address.

The server 202 may update the quality model and rules set 310 for an expiration alert 405 when the sensor data is received from the food monitoring labels. The quality model and rules set 310 for an expiration alert 405 may be used to estimate the remaining quantity of food 205 and the time remaining before the food 205 is fully consumed, reaches the threshold quantity, or spoils. The quality model and rules set 310 for an expiration alert 405 may also be generated by the server 202 retrieving information regarding prior consumption data for the food target 205 from the consumer's account. The server 202 may accomplish this using information manually entered by a consumer or from information recorded during past monitoring events and associated with the consumer's account with the server 202.

FIG. 8 is a block diagram illustrating components of a hub 201 configured to communicate with and manage food monitoring labels. The hub 201 is powered by a powering unit 803 such as a wall outlet, USB port, or rechargeable battery. The hub 201 may include an over-the-air connection such as a Bluetooth Low Energy module (BLE) 801 that interacts with a food monitoring label over a BLE protocol, a Wifi module 802, a LED 804, an actuator or user interface such as a button 805 and a micro-processor 806 for processing data packets being communicated between the hub 201, the server 202, and the labels. The BLE module 801 may be used to communicate with multiple labels to collect their sensor inputs, as well as forward any commands to the labels as instructed by the server 202. The Wifi module 802 may be configured to implement 802.11 standards to enable the hub 201 to connect wirelessly to the server 201 over TCP/IP. The components of the hub 201 may be interconnected via a bus 807.

The hub micro-processor 806 facilitates communication between the labels and the server 202 using the hub 201 as a mid-point, by receiving and processing data through the BLE module 801, the Wifi Module 802, or a hardline connection, and formatting and forwarding the data to either the labels or the server 202 depending on where the data originated. The micro-processor 806 may implement transportation security protocols such as TLS/SSL between the hub 201 and the server 202 and maintains an SSL session for future communication. The hub 201 may also implement secure communication between itself and each label. When the sensor data is received by the hub 201, the data may be buffered and stored in a memory 808 instead of being forwarded to the server 202 immediately. To save communication bandwidth and server 202 operating cost, the hub 201 may be configured to only report to the server 202 once during a pre-set time period, or when a special event occurs such as detecting motion of the food target 205 or a deviation from the ideal environmental conditions of the food target 205. The hub 201 may include a LED 804 to indicate its operational status, as well as a user interface or actuator such as a button 805 for allowing the consumer to turn on/off the hub 201 or entering the hub 201 into Bluetooth pairing mode.

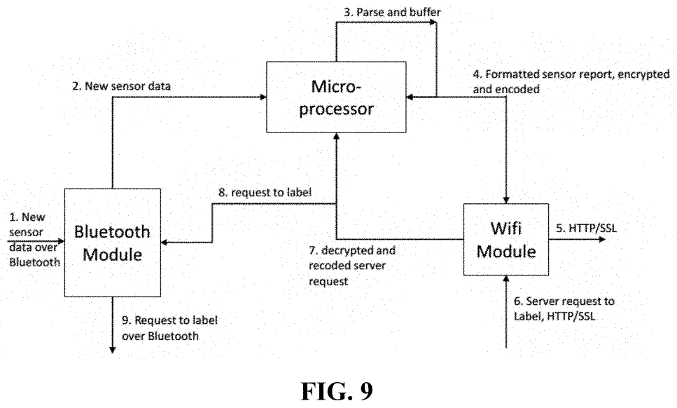

FIG. 9 illustrates an example embodiment of a process flow diagram detailing a method for a system to monitor food conditions using the hub 201. It should be understood by those skilled in the art that while the following method details an order of steps, this is only an example and the steps recited in the method may be executed in any order and is not limited to the order presented. Additionally, while the following steps detail the use of a BLE connection between the label and the hub 201, it should be appreciated by one skilled in the art that other connection methods may be used without deviating from the disclosure. At step 1, the Bluetooth module 801 in the hub 201 receives a new BLE packet containing sensor data from a food monitoring label. In step 2, the Bluetooth module 801 decodes the packet and forwards the new sensor data to the micro-processor 806. In step 3, the micro-processor 806 parses the data and buffers it under the unique identifier of the originating label. If this set of sensor data is significantly different from the previous data received, i.e. indicating variation of key environmental indices and/or motion events, the sensor data may be forwarded to the server 202 immediately, as shown in step 5. Otherwise, the data may only be buffered temporarily and the entire buffer will be forwarded at a later time. If the micro-processor 806 decides to send any data to the server 202, it may, at step 4, format the data into a report according to a server formatting rule, and perform encoding and necessary encryption. At step 5, the report is sent to the server 202 via the Wifi Module 802 over TCP/IP securely, such as HTTP/SSL.

When the server 202 wants to communicate with a label, it may send a command including the intention to communicate and any auxiliary data first to the Wifi module 802 of the hub 201, via HTTP/SSL for example, as shown in step 6. The Wifi module 802 may perform decryption and decoding before forwarding the request and data to the micro-processor 806 at step 7. The micro-processor 806 re-formats the request according to the destination label's formatting rule so the label can understand and interpret the server request. The micro-processor 806 then sends the formatted request to the Bluetooth module 801 at step 8. Finally, the Bluetooth module 801 forwards the request to the corresponding label over a Bluetooth protocol at step 9.

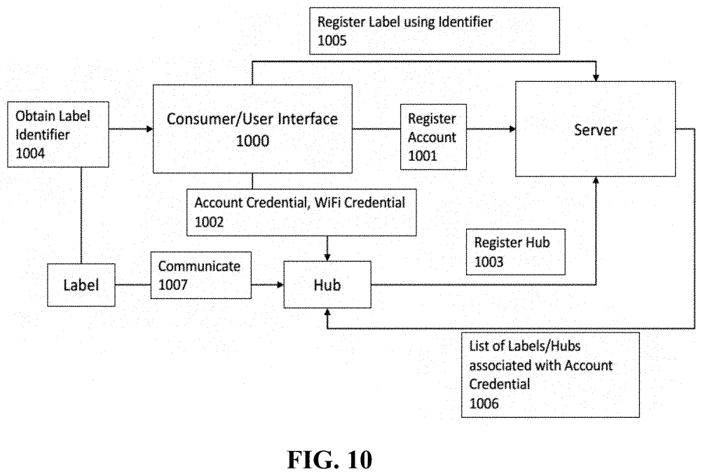

FIG. 10 illustrates an example of a provisioning model for pairing labels with a hub 201. The provisioning model allows a consumer to specify a list of labels that the consumer uses by utilizing a smart phone or Internet connected device 200. The list may then be synchronized with the server 202 and the hub 201. The smart phone or Internet connected device 200 has a connection with the server 202 either by way of a network or a physical connection. The system can support at least one hub 201 and may be configured to support multiple hubs for improved coverage and reliability when communicating with multiple labels.

In the process of provisioning a label to a hub 201, the label and the hub 201 may need to be registered by a consumer in order to communicate with each other. Each consumer may engage a user interface 1000 to create an account 1001 with a unique identifier 1002 which may be stored in a server's state database 301. Each hub 201 or label is uniquely identified with a string such as their MAC address or a unique service identifier (UUID) 1004 and 1005 respectively. The process for a consumer to register a hub 201 or a label is hereinafter referred to as provisioning.

Provisioning is the association of a consumer's account 1001 in the system with a list 1006 of identifiers for hubs 201 and labels stored in the server's 202 database. A consumer may create an account 1001 with the server 202 by registering. Following registration, a consumer may then connect with a hub 201 wirelessly from a smartphone or a device 200 using the consumer's account 1001. The consumer can connect the hub 201 to a local area network using a smartphone or Internet connected device 200 or the hub 201 using a user interface 1000. This may be accomplished by sending the network's Wifi access credentials as well as the consumer's account 1001 credentials to the hub 201. Alternatively, the consumer may input the credentials into the hub 201 using the user interface 1000.

Following this, the hub 201 is able to connect to the Wifi network and Internet, and authenticate/register 1003 with the server 202 to the consumer's account 1001. The server 201 maintains a list of labels and hubs 1006 registered to the consumer's account 1001. The list 1006 is initially empty when a consumer first registers an account 1001 but grows and shrinks as the consumer provisions the labels and hubs 201 to the consumer's account. A consumer may also de-provision and remove a registered label or hub from the consumer's account. Consumers may perform these actions on their smartphones or Internet connected device 200 using a user interface 1000 which may be implemented as an application. The hub 201 periodically synchronizes with the server 202 to obtain the latest list 1006 of labels that the hub 201 needs to track and monitor.

Each label has a unique identifier 1004 such as its Bluetooth Mac address or service UUID. When a label communicates with the hub 201, this identifier is also sent 1007. The hub 201 uses the identifier to associate any received sensor data from the label with a registered consumer account. The consumer may provision a label, using the label's unique identifier obtained 1004 from the label or its packaging. Alternatively, a label may be provisioned using the consumer's account identifier. This may be done using the consumer's smartphone or device 200. This process may be fulfilled by a consumer typing the identifier found on the label's factory packaging, scanning the identifier in via barcode or QR code, or connecting to and communicating with the label first and then reading out the identifier 1004 wirelessly or through a hardline connection. Once the label's identifier is obtained 1004, the consumer may submit the identifier to the server 202, along with the consumer's account identifier for adding the label to the list of the consumer's labels and hubs 1006.

This process may be completed using a smartphone or device 200 with an associated application loaded onto it. After the server 202 authenticates the provisioning request, the label's identifier will be used to add the associated label to the list of labels and hubs 1006 under the consumer's account. For convenience, it may be possible to provision multiple labels in a kit/box at the same time by obtaining an identifier 1004 associated with the entire kit/box, or by reading an identifier of any one of the labels in the same box/kit. When this identifier is received by the server 202, the server 202 may register all the labels in the same box/kit using the unique identifiers for each label in the box/kit and adding all of the labels to the consumer's account list 1006. Additionally, a label may be registered to a joint account for use with multiple consumers. In such an event each consumer has an individual identifier which can be loaded onto the label when beginning a monitoring session. This will allow consumers with communal refrigerators 206 and the like to use a shared set of labels without having to re-provision and de-provision the label prior to each monitoring session.

While monitoring, the labels may continuously send sensor data from the label's sensors 705 to the hub 201. The hub 201 may then forward this information to the server 202. This information may include the target food's 205 holding environment parameters, signs of deterioration, and information on movement and consumption of the food target 205.

The server 202 may maintain a model that tracks the quality and consumption level for each target food 205 in the food storage system 206. The sensor data received from the hub 201 is used to update and maintain accuracy and consistency of the model. As discussed in detail in describing the structure and methods hereinbefore, the model can be used to predict food spoilage as well as the food consumption level using a rules set 405. If food deterioration or spoilage is detected, an alert 406 may be sent to the consumer's smartphone or device 200. The alert 406 may also be sent to the labels themselves, via the hub 201. The labels, upon receiving an alert 406, may indicate the alert by turning on an LED 706 with a color, and/or ringing a buzzer 609. These visual/audio indicators may be configured to only turn on when the consumer is in close proximity to the labels and food target 205, such as when the consumer opens the doors of the food storage system 206. The labels may have a mechanism for detecting movement using various sensors 603. For example, changes in ambient light may be an indication of the consumer opening or closing the food storage system 206. A pressure sensor or accelerometer may also be used to detect movement of the food or its container 205 as these events are typically correlated with consumer presence. The visual/audio indications provide instant, on-the-spot feedback to the consumer while the application of proximity sensors improves power consumption of the system.

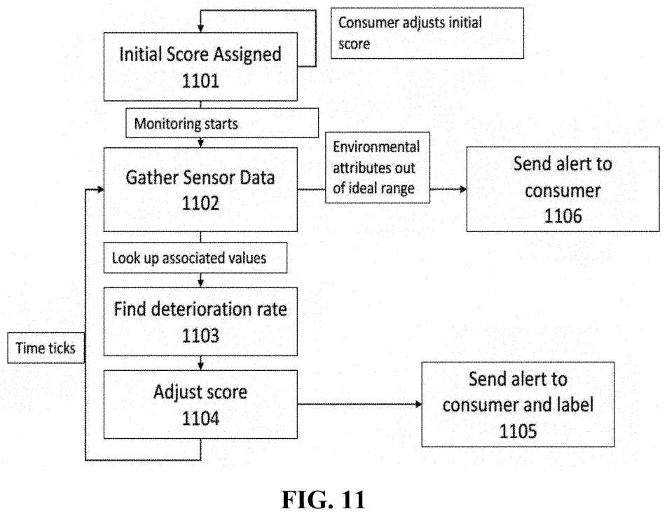

FIG. 11 illustrates a method of forming and updating a quality model and rules set 310 for an expiration alert 1106. During monitoring, each label may continuously collect sensor data 1102 regarding the holding environment, consumption, and deterioration of the food target 205. Alternatively, the label may be configured to take readings at timed intervals or in response to an external stimulus. The label may then share the information with the server 202. The label may also share the information with the hub which then shares it with the server, and alternatively, the label may transmit information to one or the other, or both the server 202 and the hub 201.

In an embodiment, the server 202 maintains a model for each food 205 that is being monitored. From implementation and processing standpoint it is desirable to simplify the label and hub 201 because they both run on embedded processors. The primary function of the label(s) and hubs are to collect and convey sensor data to the server (and convey commands from server to labels in the reverse direction). The server does all the heavy processing: updating and maintaining a tracking and depletion model for an active tracking session of a plurality of foods, detecting deviation of ambient environment condition(s) of the food holding/storage, issuing warnings, performing depletion prediction processing, etc. The hub may serve as a local server in the local area network.

The tracking or food quality model may be designed to imitate and keep track of the deterioration rate 1103 of the food target 205, using sensor data received regarding variables for food quality such as food type, length of time in the food storage system, ambient temperature in the food storage system, consumption events and their frequency, as well as data for similar food types from past monitoring sessions. The tracking/quality model may be configured to make predictions, based on some intelligent rules set 310, to estimate food expiration date, alert a consumer 1106 about deviation in the environmental conditions of the food storage system, prepare an automatic shopping list, and place an order for replenishing and restocking food based on historical data, habitual preference, and consumer input.

In one embodiment of a quality model and rule set for an expiration alert 1106, a scoring function is parameterized and used. When a label begins monitoring, an initial score 1101 is assigned to the food target 205 based on its initial level of freshness and quality. A default score 1101 may be set and can be adjusted by the consumer manually. As the holding time elapses, the adjusted score 1104 is deducted according to the rules set 405 per every unit amount of time. At the beginning of each time unit, the latest set of sensor observations such as temperature, light exposure, or humidity are obtained and reviewed 1102. If any of these environmental attributes are outside an ideal range, an alert 1106 may be sent to the consumer. A dictionary 305, such as may be implemented in a look-up table, may be used to look-up and obtain the attributes of the food target 205 and estimate the current deterioration rate 1103 from the sensor data. The deterioration rate 1103 may be applied to adjust the score 1104.

The following pseudo-code illustrates a scoring function update, according to the disclosure, in continuous tracking of a food type, in this example a bottled milk (with 5 minutes resolution for model advancement):

Assign initial score to the tracking target: Default is 100 but allows consumer to manually override for a lower score.

While(1){
If user stops tracking
   Break out of while-loop and end tracking session

Advance time by 5 minutes

Gather sensor inputs such as ambient temperature, light condition and Ph level
If ambient temperature, light condition or Ph level is out of optimal range for Milk
   Send Alert to consumer for attention

Lookup dictionary for a new “Deterioration rate” based on time and new sensor data

Score is deducted by “Deterioration rate” *5 minutes

If new score is too low
Send alert to consumer
Send alert to Label for LED and Buzzer indication

Shelf life remaining is predicted as

Score remaining/“Deterioration rate”}

The score may be measured in a range divided into several regions, with each region representing a level of food quality, for example, fresh, not-so-fresh but consumable, and expired. A set of threshold scores are defined characterizing these regions. If a decrease in food quality level is detected, an alert 1106 may be sent to a consumer's smartphone or device 200. Likewise, a signal 1105 may be sent to the corresponding label for the label to notify the consumer through audio and visual indicators reflecting the current food quality level. The signal 1105 may also be sent to the consumer's smartphone or device 200. The system repeats the process as time advances, or until the label's monitoring session ends.

In an illustrative embodiment, a label may be specifically configured for monitoring a beverage such as milk and may include a temperature sensor, a pH level (Acidity) sensor, as well as a photoelectric sensor. The temperature sensor may continuously measure and report on the storage temperature of the milk, since deviations from the optimal storing temperature may have a significant impact on the milk's shelf life. The pH acidity sensor may take a more direct measurement regarding the milk's deterioration level as increased acidity typically indicates poor milk quality and high levels of deterioration. Additionally, photoelectric sensors measure the amount and intensity of light the milk has been exposed to during storage as prolonged exposure to strong light may also decrease the milk's shelf life.

In another illustrative embodiment, a label may be specifically configured for monitoring fresh fruit and may include a temperature sensor, a humidity and water sensor, a color sensor and optionally an advanced gas sensor array. Temperature is a significant factor on a fruit's expiration time. Humidity and water sensors offer further indicators on when the fruit will expire. The color sensor can measure and track the changes to the exterior color of the fruit due to deterioration. The label uses the sensor data to provide a server 202 with information indicating the progression of the monitored food's deterioration. Gas sensors, measuring and identifying the presence of a specific set of analytes such as ethanol, methanol, acetic acid and CO2 may further improve the accuracy of the predictive model the server 202 uses for food expiration prediction by providing additional indicators of the food's deterioration rate.

Another illustrative embodiment may allow the food monitoring system to be integrated with a food storage system 206. By way of illustration, a localized hub may be within a refrigerator such that the localized hub can function as described hereinbefore and communicate with any labels inside the refrigerator while interacting with the refrigerator 206 to ensure optimal conditions within the food storage system 206 for the contained food targets 205. The refrigerator may include a user interface 805 on an exterior side or front panel for consumer interaction.

The food storage system 206 in this example may have an integrated hub as described with reference to FIG. 8, and may house an internal BLE module 801 for communicating with the labels. The powering unit 803 may be the wall connector or another powering method used by the food storage system 206. The micro-processor 806 may be a special configuration designed for use with the food monitoring system. The user interface 805 of the food storage system 206 may use actuators or a touch screen display. Additionally, a LED 804 may be integrated into the user interface 805 to display operational status, system alerts, and/or food quality.

In an illustrative embodiment, the method of beginning a monitoring session may include programming a label through the user interface 805 of the food storage system 206 to select the type of food 205 to be monitored. A label storage panel may then be configured to illuminate the label programmed for the food, or a pre-defined label selected from the user interface 805 corresponding to the food 205 to be monitored. The consumer may then remove the label from the label storage panel. The food 205 and the label may be stored in the refrigerator 206 where a monitoring session then takes place. The removal of the label from the label storage panel may signal the label to begin a monitoring session, additionally storing the label in the storage panel may signal the label to end the monitoring session.

The label sends sensor data to the hub 201 where it may then be forwarded to a server 202 in real time or at timed intervals. The server 202 can then be accessed by the consumer from a smartphone or device 200, the user interface of the hub 805, or by logging into their account with the server 202. Additionally, the consumer may verify food quality and quantity via the user interface 805 of the hub 201 prior to opening the food storage system 206. Alternatively, the label's LED 706 may be coded to light up and the buzzer 609 to ring only when the food storage system 206 is opened or upon detecting a consumer in proximity to the food storage system 206.

Alternative embodiments may include integrating the hub 201 with a food storage system 206 such as a food pantry, refrigerator, or a closet. In such embodiments, the label may be specially configured to operate optimally in the conditions such a storage system 206 presents to ensure the label is best able to complete the monitoring session.

Although the embodiments disclosed hereinbefore have described a partitioning of operations and functions between a hub and a server, one skilled in the art should appreciate that the functionality of the hub and/or the server may be divided up in a different manner for example operations may be interchangeable such that the operations and functions of the server may be performed by the hub and vice versa. Additionally, one skilled in the art should appreciate that the hub and the server could be combined in a single processor (distributed or centralized) configured to perform the operations and functions described hereinbefore.

The detailed description set forth herein, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

Based on the teachings, one skilled in the art should appreciate that the scope of the present disclosure is intended to cover any aspect of the present disclosure, whether implemented independently of or combined with any other aspect of the present disclosure. For example, an apparatus may be implemented or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure may be embodied by one or more elements of a claim.

The words “exemplary” or “illustrative” are used herein to mean “serving as an example, instance, or illustration.” Any aspect described herein as “exemplary” or “illustrative” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses or objectives. Rather, aspects of the present disclosure are intended to be broadly applicable to different technologies, system configurations, networks and protocols, some of which are illustrated by way of example in the figures and in the following description of the preferred aspects.

Although illustrative embodiments of the present disclosure have been described herein with reference to the accompanying drawings, it is to be understood that the present disclosure is not limited to those precise embodiments, and that various other changes and modifications may be made by one skilled in the art without departing from the scope or spirit of the disclosure. The detailed description and drawings are merely illustrative of the present disclosure rather than limiting, the scope of the present disclosure being defined by the appended claims and equivalents thereof.

What is claimed is:

1. A system for monitoring food quality comprising:
a food monitoring label including at least one sensor, and a processor specially configured to receive a sensor data from the at least one sensor;
a hub comprising a hub processor specially configured to receive the sensor data from the food monitoring label, and a user interface configured to display information relating to the sensor data;
a server in communication with the hub comprising a server user interface configured to issue instructions to the food monitoring label and view the sensor data and information relating to the sensor data.

2. The system for monitoring food quality of claim 1 wherein the food monitoring label further comprises a fastener to affix the food monitoring label to a food to be monitored by the food monitoring label.

3. The system for monitoring food quality of claim 1 wherein the processor of the food monitoring label is configured to encode the sensor data and the hub processor is configured to decode the sensor data.

4. The system for monitoring food quality of claim 3 wherein the hub processor is configured to format the instructions to be readable by the processor of the food monitoring label, and the hub is in communication with the food monitoring label to send the formatted instructions to the food monitoring label.

5. The system for monitoring food quality of claim 1 wherein the food monitoring label is configured to monitor a food for at least one sign of deterioration in the food and the at least one sensor is selected from a group consisting of a temperature sensor, a humidity sensor, a CO2 sensor, a Total Volatile Organic Carbon sensor, a photoelectric sensor, a color sensor, a water sensor, an optical sensor, a biochemical sensor, an accelerometer, a hall effect sensor, a proximity sensor, a touch sensor, a pressure sensor, a gyrometer, a gyroscope, a compass, a laser sensor, an infrared sensor, and an ultrasonic sensor.

6. The system for monitoring food quality of claim 2 wherein the hub further comprises a Bluetooth module configured to pair with the food monitoring label and receive the sensor data from the food monitoring label, the Bluetooth module being operatively connected to the hub processor to send the sensor data to the hub processor.

7. A method of monitoring food quality comprising:
loading a food information specific to a food onto a food monitoring label comprising at least one sensor operatively connected to a processor specially configured to receive a sensor data from the at least one sensor;
affixing the food monitoring label to the food;
instructing the food monitoring label to begin a monitoring session of the food using the at least one sensor to monitor a quality of the food selected from the food information, the quality of the food being measured as the sensor data from the at least one sensor;
generating an alert when the sensor returns the sensor data indicating the food quality has changed;
transmitting the alert to a server recording the sensor data and using the sensor data to predict a time of expiration of the food;
updating the server's prediction of the expiration time of the food using the sensor data.

8. The method of monitoring food quality of claim 7 further comprising:
pairing the food monitoring label with a hub comprising a hub processor operatively connected to a user interface disposed on an exterior of the hub;
the hub communicating with the food monitoring label to send and receive instructions and the sensor data;
the server retaining a list comprising a unique identifier of the food monitoring label paired with the hub;
the hub associating the sensor data with the unique identifier of the food monitoring label;
the hub processor forwarding the sensor data to the server;
the server storing the sensor data associated with the unique identifier of the food monitoring label;
the server generating instructions for the food monitoring label and forwarding the instructions to the hub;
the hub receiving the instructions and the hub processor formatting the instructions to be readable by the processor of the food monitoring label;
the hub forwarding the formatted instructions to the food monitoring label;
the food monitoring label receiving the formatted instructions and the processor of the food monitoring label reading the instructions.

9. The method of monitoring food quality of claim 7 wherein:
the food monitoring label is further configured to monitor the quantity of the food in a food storage system using the at least one sensor;
the at least one sensor reporting the quantity of the food in the sensory data;
the server predicting when the food quantity will reach a threshold amount;
generating an alert when the sensor data indicates the food quantity has reached the threshold amount.

10. The method of monitoring food quality of claim 8 further comprising:
the server predicting the expiration time of the food using the sensor data;
generating an alert if the food is predicted to be expired, the alert indicating a food status selected from a group consisting of fresh, not-so fresh, and expired;
forwarding the alert to a consumer;
sending the alert to the hub, the hub further comprising a LED;
the hub indicating the food status through a color associated with the selected food status.

11. The method of monitoring food quality of claim 8 further comprising the hub processor encoding and decoding the sensor data to be readable by the server.

12. The method of monitoring food quality of claim 9 further comprising forwarding the alert to a food merchant, the alert further comprising an order to replenish and restock the food.

13. The method of monitoring food quality of claim 10 wherein the alert further comprises a prompt for the consumer to place an order with a food merchant to replenish and restock the food.

14. A food monitoring label comprising:
at least one sensor in communication with at least one processor;
the at least one processor being specially configured to receive a sensor data generated by the at least one sensor and prepare the sensor data to be transmitted through an antenna disposed on the food monitoring label.

15. The food monitoring label of claim 14 wherein the food monitoring label is configured to monitor a food for at least one sign of deterioration in the food and the at least one sensor is selected from a group consisting of a temperature sensor, a humidity sensor, a CO2 sensor, a Total Volatile Organic Carbon sensor, a photoelectric sensor, a color sensor, a water sensor, an optical sensor, a biochemical sensor, an accelerometer, a hall effect sensor, a proximity sensor, a touch sensor, a pressure sensor, a gyrometer, a gyroscope, a compass, a laser sensor, an infrared sensor, and an ultrasonic sensor.

16. The food monitoring label of claim 14 wherein the at least one sensor is configured to monitor a food during a monitoring session, the sensor data comprising a food quality data and a food quantity data generated by the at least one sensor during the monitoring session of the food.

17. The food monitoring label of claim 14 further comprising a fastener disposed on an exterior of the food monitoring label configured to affix the food monitoring label to the food to be monitored.

18. The food monitoring label of claim 16 further comprising a LED configured to display a color associated with a food status determined by the at least one processor based on the food quality data.

19. The food monitoring label of claim 16 further comprising a user interface disposed on an exterior of the food monitoring label, the user interface being configured for a consumer to send instructions to the at least one processor and the at least one sensor to begin the monitoring session of the food.

20. The food monitoring label of claim 17 wherein the fastener is selected from a group consisting of an alligator clip, a suction cup, hook and pile, a magnet, a retractable leash, at least one hook, and an adhesive.
